SEQUENCE LISTING (20 sequences)

SEQ ID NO: 1 - protein, 10 aa, synthetic construct
GFTFSDYYMY

SEQ ID NO: 2 - protein, 17 aa, synthetic construct
YINSGGGSTYYPDTVKG

SEQ ID NO: 3 - protein, 10 aa, synthetic construct
RGLPFHAMDY

SEQ ID NO: 4 - protein, 11 aa, synthetic construct
SASQGISNYLN

SEQ ID NO: 5 - protein, 7 aa, synthetic construct
YTSILHS

SEQ ID NO: 6 - protein, 9 aa, synthetic construct
QQFNKLPPT

SEQ ID NO: 7 - protein, 284 aa, Mycobacterium tuberculosis
SRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 8 - protein, 97 aa, Mycobacterium tuberculosis
TSMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 9 - protein, 206 aa, Mycobacterium tuberculosis
AAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 10 - protein, 121 aa, synthetic construct
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSAS

SEQ ID NO: 11 - protein, 103 aa, synthetic construct
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTK

SEQ ID NO: 12 - protein, 446 aa, synthetic construct
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 214 aa, synthetic construct
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 27 aa, synthetic construct
QTPTNTISVTPTNNSTPTNNSNPKPNP

SEQ ID NO: 15 - protein, 25 aa, synthetic construct
SSVSPTTSVHPTPTSVPPTPTKSSP

SEQ ID NO: 16 - protein, 25 aa, synthetic construct
PTSTPADSSTITPTATPTATPTIKG

SEQ ID NO: 17 - protein, 25 aa, synthetic construct
TVTPTATATPSAIVTTITPTATTKP

SEQ ID NO: 18 - protein, 25 aa, synthetic construct
TNGSITVAATAPTVTPTVNATPSAA

SEQ ID NO: 19 - protein, 649 aa, synthetic construct
SRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGEFPTSTPADSSTITPTATPTATPTIKGTSMTEQQWNFAGIEAAASAIQGNVTSIHSLLDEGKQSLTKLAAAWGGSGSEAYQGVQQKWDATATELNNALQNLARTISEAGQAMASTEGNVTGMFAKLGGAPTNGSITVAATAPTVTPTVNATPSAASSAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAEF

SEQ ID NO: 20 - protein, 1126 aa, synthetic construct
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGEFPTSTPADSSTITPTATPTATPTIKGTSMTEQQWNFAGIEAAASAIQGNVTSIHSLLDEGKQSLTKLAAAWGGSGSEAYQGVQQKWDATATELNNALQNLARTISEAGQAMASTEGNVTGMFAKLGGAPTNGSITVAATAPTVTPTVNATPSAASSAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAEF

(19) United States
(12) Patent Application Publication
(10) Pub. No.: US 2026/0226183 A1
(43) Pub. Date: Aug. 6, 2026

(54) ANTI-TUBERCULOSIS VACCINE TARGETING SELECTED MYCOBACTERIUM TUBERCULOSIS PROTECTIVE ANTIGENS TO DENDRITIC CELLS

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); UNIVERSITÉ PARIS-EST CRÉTEIL VAL DE MARNE, Creteil (FR); BAYLOR RESEARCH INSTITUTE, Dallas, TX (US)

(72) Inventors: Yves LEVY, Creteil (FR); Gérard ZURAWSKI, Dallas, TX (US); Sandra ZURAWSKI, Dallas, TX (US); Sylvain CARDINAUD, Créteil (FR); Christine LACABARATZ, Créteil (FR); Patrick LECINE, Vitry-Sur-Seine (FR); Nora KAKWATA-NKOR, Orsay (FR)

(21) Appl. No.: 19/149,612

(22) PCT Filed: Feb. 1, 2024
(86) PCT No.: PCT/EP2024/052500

§ 371 Date: Jul. 21, 2025

(30) Foreign Application Priority Data
Feb. 2, 2023 (EP) 23305134.1

(51) Int. Cl. C07K 16/28; A61K 39/04; A61P 37/04; C07K 14/35

(52) U.S. Cl. CPC C07K 16/2878; A61K 39/04; A61P 37/04; C07K 14/35; C07K 2317/24; C07K 2319/33

(57) ABSTRACT

There is an urgent need for an efficient therapeutic vaccine against tuberculosis (TB), which remains a major public health issue. Current “classic” strategies under development failed or are not optimal, and to reach the World Health Organization's 2035 End TB Strategy, more efficacious vaccines are needed. The inventors have generated a post-exposure/therapeutic TB vaccine candidate (CD40.TB), consisting in an antibody directed against a surface antigen (i.e., CD40) of an antigen presenting cell (i.e., dendritic cell) wherein the heavy chain is conjugated to 3 pertinent Mycobacterium tuberculosis (Mtb) antigens, and prone to induce strong anti-TB humoral and cellular immunity.

### FIELD OF THE INVENTION

The present invention is in the field of medicine, in particular infectious diseases.

### BACKGROUND OF THE INVENTION

TB remains a major global human health crisis, which even worsened due to reduced preventive diagnosis during the COVID-19 pandemic, accounting for 1.5 million deaths in 2020 according to the WHO Global Tuberculosis Report 2021. It is thought that 5-10% of individuals exposed to Mtb develop active TB disease [1]. Although displaying immune reactivity to Mtb, the majority of Mtb-infected individuals remains asymptomatic, and either clears the pathogen or maintains a latent infection (LTBI). In patients with active TB disease, the poor sensitivity of Mtb to anti-TB drugs is one cause of the prolonged duration of TB treatment. Indeed, the current guidelines recommend a 6-month regimen for patients with active drug susceptible TB, which consists of 2 months of therapy with a 3-drug regimen followed by an additional 4 months with a 2-drug regimen. The current TB treatment is associated with several issues, in particular a significant toxicity due to the long duration of the treatment, a poor adherence to treatment leading to the emergence of antimicrobial resistance and ongoing risk of transmission, especially in patients with high bacterial burden. Multi-drug resistant (MDR)-TB cases, with 84 countries reporting at least one case of extensively-drug resistant (XDR)-TB in 2018 [2], represent a continuous major threat to human health, despite encouraging advances with new drug combinations [3], which however, may show considerable toxic effects. In this context, post-exposure/therapeutic anti-TB vaccines may constitute a pertinent strategy to help shortening the risk of secondary transmission during initial treatment, to avoid drug toxicity through reduction of treatment duration, and overall, to prevent reactivation of TB disease in LTBI individuals.

While clinical evidence suggests that natural immunity may control TB infection in some individuals, this phenomenon is encountered in only a few patients [4]. Following upper airways infection, Mtb infects macrophages in lungs using several components of their membrane as receptors, and manipulates them to decrease apoptosis in order to promote their survival [5]. Infected macrophages promote IL-10 and IFN-γ responses while decreasing the membrane expression of their MHC and co-stimulatory molecules [6]. These negative responses delay CD4+ T cell priming and homing to the lung. However, these initial Th1 responses slow down the infection and prevent its dissemination through the formation of granulomas [7]. While also involved during primary response, CD8+ T cells seem to play a less important role during the initial phase of TB infection.

The importance of T-cell immunity in the control of Mtb infection has been demonstrated in human and non-human primates (NHP) models. Defects in Th1 cytokine production (namely IFN-γ), or higher risk of progression from latent to active TB in immunodeficient HIV infected patients, highlight the role of T cell responses in controlling TB disease progression in humans [8]. Interestingly, it has been shown that latent and active TB differ in term of T cell responses, active TB being associated with elevated frequencies of Mtb-specific CD4+ T cells with single or dual function (TNF-α+ or TNF-α+ IFN-γ+), while tri-functional (IFN-γ+ TNF-α+IL2+) Mtb-specific CD4+ T cells are more frequently detected during latent infection [9]. Change in CD8+ T cell phenotype and function have been also described during the transition from latent to active TB, with a higher frequency of Mtb-specific CD8+ T cells in active TB patient but these cells seem to be less functional and differentiated as compared to LTBI [8,9]. Finally, the involvement of IL-17 producing cells in Mtb immunology has been highlighted. Here also, active TB is associated with a decrease in IL-17-producing cells in the blood. However, it seems that in this case IL-17-producing cells are sequestered in the lung during active disease [10]. Several transcriptomic analyses of whole blood from active TB patients or LTBI subjects have been conducted in the past few years [11-13]. Signature of active TB is clearly dominated by overexpression of IFN type I and II-inducible genes and those related to myeloid or inflammatory cells, and by downregulation of genes encoding B and T cell functions.

T cell responses therefore appear to be the most relevant target for an effective anti-Mtb therapeutic vaccine, and differences observed in their functions and/or phenotype in latent and active TB is of crucial importance, indicating which “adequate” T cell responses have to be induced by an efficient therapeutic vaccine for preventing reactivation of TB disease in LTBI individuals.

Currently, Bacillus Calmette-Guérin vaccine (BCG) is the only available vaccine against TB showing variable efficacy in reducing the incidence of pulmonary TB in adults and adolescents [14]. BCG is a live attenuated strain of Mycobacterium bovis, which in particular lacks the so-called Region of Difference 1 (RD1) including genes encoding the elements of the Type VII Secretion System “ESX-1” which exports major Mtb virulence factors including the Early Secreted Antigenic Target, 6 kDa (ESAT-6) and Culture Filtrate Protein, 10 kDa (CFP-10). BCG induces both protective and anti-protective responses that may contribute to TB persistence. Although correlates of protection and/or TB immune control are not established to date, it is widely accepted that an effective TB vaccine must elicit Ag-specific appropriate effector CD4+ (Th1) and CD8+ T cells.

So far, fourteen vaccine candidates are in clinical trials, half of them have been evaluated in therapeutic regimen, and some of them are in phase II/III [14-16]. Two different strategies based on whole mycobacterial cell-derived vaccines, i.e. live-attenuated or heat killed bacteria, or subunit vaccines are developed. Therapeutic vaccine strategies using inactivated mycobacterium species such as Mycobacterium indicus pranii or Mycobacterium vaccae have been associated with some degree of efficacy both in murine models and in human [17]. Efficacy of these two strains correlates with an enhancement of T cell immunity, and a modulation of the inflammatory response that prevents extracellular bacterial growth. The RUTI vaccine is also another therapeutic vaccine that has an impact on both replicating and non-replicating Mtb [18]. Alternative strategies were developed on the basis of a down-selected repertoire of Ags (protein-based vaccines formulated with adjuvant), focusing on actively secreted mycobacterial proteins involved in TB pathogenesis, namely ESAT-6, CFP-10, TB10.4, Apa, proteins of the Ag85 complex, Mpt64, Mtb19, Mtb32, Mtb39 or proteins of the PE/PPE superfamilies [19-21] expressed at different stages of infection/disease. Some of these Ags gave promising results in animal models and humans [14, 17, 22-28]. One group used Mtb proteins involved in iron sequestration, as well as Ags produced by the bacterium under hypoxic conditions (Rv1738, Rv1909, Rv2032, Rv2359, Rv2711, Rv3130, and Rv3841). A single dose of this vaccine was unable to decrease bacterial load in the lungs of mice but increased the animals' survival after boost [25]. In contrast, the ID93 vaccine, comprising non-replicating (Rv2608, Rv3619, Rv3620) and replicating Mtb Ags (Rv1813), was able to induce both immunological and bacteriological responses in mice and NHP models of TB [26]. More recently, 8 protective antigens were selected to create an Mtb-specific subunit vaccine, named H107, which when co-administered with BCG, leads to increased adaptive responses against both H107 and BCG and broadened the overall vaccine repertoire with Th17 responses and less-differentiated Th1 cells [27]. The evolution of vaccine technology allowed development of DNA vaccines, whose protective potential was initially controversial. However, it seems that when given with anti-TB drugs, DNA vaccines encoding either Ag85B or Hsp65 showed compelling evidence of therapeutic activity [28]. Viral vector vaccines (poxvirus and adenovirus) developed for a prophylactic purpose gave modest efficacy in NHP models, and notably the MVA: Ag85A vaccine failed to demonstrate any clinical benefit in humans [29]. Recently, the team of L. Picker developed a RhCMV-based TB vaccine and immunized NHP with the 68-1 RhCMV strain, known to induce unconventional CD8+ T cell responses restricted by MHC-II and MHC-E molecules, expressing 9 different Mtb proteins: Ag85A, Ag85B, ESAT-6, Rv3407, Rv1733, Rv2626, RpfA, RpfC and RpfD [30]. A 40% protection was observed, and in an additional 30% of the animals, the vaccine helped to control infection, demonstrating both a prophylactic and a therapeutic effect. Although encouraging, whether this strategy can be used to design a vaccine deployable in human remains to be determined. Indeed, while RhCMV has considerable homology with human CMV, it is a replication-competent virus, which could be potentially pathogenic in vulnerable people and it is not obvious whether an attenuated or replication-defective vector could elicit the type of T cell responses able to mediate sterilizing immunity against Mtb in human. GamTBvac, one of the 4 vaccines in phase III clinical trial, is a recombinant subunit TB vaccine containing Ag85A and ESAT6-CFP10 antigens and CpG ODN adjuvant formulated with dextrans [31]. Another promising vaccine candidate in human is the M72/AS01E vaccine containing a recombinant fusion protein derived from the Mtb32A and Mtb39A Ags and a liposome-based adjuvant, initially developed by GSK for a prophylactic purpose. Its therapeutic properties were also explored in a phase IIb trial in infected individuals without any sign of active TB, and data showed that 2 administrations of the vaccine elicited an immune response and provided 49.7% protection against progression to pulmonary TB disease in a 3-year follow-up trial [32]. This prophylactic vaccine will soon enter phase III clinical trial.

Globally, these data highlight that: i) even if the composition of the most seemingly protective Ags remains an unresolved question in TB vaccinology, previous studies have identified several relevant T cell Ags to include in therapeutic vaccines. These comprise Ags which are actively secreted via diverse secretion systems of Mtb, and notably associated with the ESX-1 Type VII secretion system involved in Mtb survival in the host and expressed at high levels during both acute and chronic phases of infection, namely ESAT-6 and CFP-10, as well as other secreted Ags, such as Mpt64 and members of the Ag85 complex, and ii) the effect of the adjuvant appears to be crucial and emphasizes the need to activate the immune system for eliciting an appropriate immune response.

One way to increase immunogenicity of proteins is to improve their delivery to the Ag-presenting cells, especially dendritic cells (DC), which capture, process and present Ags to T cells as peptides bound to both major histocompatibility complex (MHC) class I and II [33-35]. In particular, WO2012129227 discloses fusion proteins with antigens for DC-targeting vaccine generation by conjugating several M. tuberculosis protein antigens with high affinity monoclonal antibodies against several DC receptors with a view to developing novel human vaccines based on in vivo DC-targeting.

### SUMMARY OF THE INVENTION

The present invention is defined by the claims. In particular, the present invention relates to anti-tuberculosis vaccine targeting selected Mycobacterium tuberculosis protective antigens to dendritic cells.

### DETAILED DESCRIPTION OF THE INVENTION

### Main Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence.

As used herein, the term “fusion protein” indicates a protein created through the attaching of two or more polypeptides which originated from separate proteins. In particular fusion proteins can be created by recombinant DNA technology and are typically used in biological research or therapeutics. Fusion proteins can also be created through chemical covalent conjugation with or without a linker between the polypeptides portion of the fusion proteins. In the fusion protein the two or more polypeptide are fused directly or via a linker.

As used herein, the term “directly” means that the first amino acid at the N-terminal end of a first polypeptide is fused to the last amino acid at the C-terminal end of a second polypeptide.

As used herein, the term “linker” has its general meaning in the art and refers to an amino acid sequence of a length sufficient to ensure that the proteins form proper secondary and tertiary structures. In some embodiments, the linker is a peptidic linker which comprises at least one, but less than 30 amino acids, e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation. The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (1) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-(H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk-see the section entitled«How to identify the CDRs by looking at a sequence»within the Antibodies pages.

As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, an agonist molecule, e.g., CD40 Ligand, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein, the term “antigen” or “Ag” has its general meaning in the art and refers to a molecule capable of being specifically bound by an antibody or by a T cell receptor (TCR) if processed and presented by MHC molecules. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “epitope” has its general meaning in the art and a fragment of at least 8 amino acids that is recognized by an immune response component. As used herein, the term “immune response component” includes, but is not limited to, at least a part of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a CD1 molecule, a B lymphocyte, an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, an antibody fragment, and/or synthetic antibody. The term “epitope” may be used interchangeably with antigen, paratope binding site, antigenic determinant, and/or determinant.

As used herein, the term “polyepitope polypeptide” refers to a polypeptide that comprises at least 2 epitopes.

As used herein, the term “Mycobacterium tuberculosis” or “Mtb” has its general meaning in the art and is a species of pathogenic bacteria in the family Mycobacteriaceae and the causative agent of tuberculosis . . .

As used herein, the term “Ag85B” refers to one protein of the Ag85 complex. The Ag85 complex is indeed a 30-32 kDa family of three proteins (Ag85A, Ag85B, and Ag85C), which all three possess enzymatic mycolyl-transferase activity involved in the coupling of mycolic acids to the arabinogalactan of the cell wall and in the biogenesis of cord factor. Ag85B contains several immunodominant T cell epitopes (Huygen K, Front Immunol. 2014; PMID: 25071781) and Ags of the Ag85 complex are part of several vaccine candidates currently being tested for TB. In particular, BCG over-expressing Ag85B confers greater protection against Mtb than BCG in guinea pigs (Horwitz M A, Proc Natl Acad Sci USA. 2000; PMID: 11095745), and DNA vaccination with plasmids encoding Ag85B conferred robust Th1 immunity and protection in mice (Lozes E, Vaccine. 1997; PMID: 9234526). Moreover, several Ag85B epitopes are recognized by human CD4+ and CD8+ T cells from PPD+ individuals (Silver R F, J Immunol. 1995; PMID: 7722319; Valle M T, Clin Exp Immunol. 2001; PMID: 11207652; Mustafa A S, Infect Immun. 2000; PMID: 10858206; Lindestam Arlehamn C S, PLOS Pathog. 2013; PMID: 23358848; Weichold F F, Genes Immun. 2007; PMID: 17429413).

As used herein, the term “ESAT-6” refers to one of the most immunogenic Ags of Mtb. This Ag is part of a number of live attenuated and subunit vaccines currently being tested for TB, including MTBVAC, (Marinova D, Exp Rev Vaccines. 2017; PMID: 28447476), H1 (Mearns H, Vaccine. 2017; PMID: 27866772) and GamTBvac (Tkachuk A P, Vaccines 2020; PMID: 33153191), but is not present in BCG

As used herein, the term “Mpt-64” refers to a secreted protein (Stamm C E, mSphere. 2019; PMID: 31167949) encoded in the region of difference RD2, which is absent from “late” BCG strains, such as the most widely used Pasteur and Danish BCG subtypes (Brosch R, Proc Natl Acad Sci USA.2007; PMID: 17372194).

As used herein, the term “tuberculosis” or “TB” has its general meaning in the art and refers to the bacterial infection caused by Mtb. TB is a potentially serious infectious disease that mainly affects the lungs. The bacteria are spread from person to person through tiny droplets released into the air via coughs and sneezes.

As used herein the term “APCs” or “Antigen Presenting Cells” denotes cells that are capable of activating T and B-cells, and include, but are not limited to, certain macrophages, B cells and dendritic cells

As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991)).

As used herein, the term “CD40” has its general meaning in the art and refers to human CD40 polypeptide receptor. In some embodiments, CD40 is the isoform of the human canonical sequence as reported by UniProtKB-P25942 (also referred as human TNR5).

As used herein, the term “subject” or “subject in need thereof”, is intended for a human or non-human mammal. Typically the patient is affected or likely to be infected with Mycobacterium tuberculosis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccination” or “vaccinating” means, but is not limited to, a process to elicit an immune response in a subject against a particular antigen.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein, the term “adjuvant” refers to a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dendritic cells (DCs).

As used herein, the expression “therapeutically effective amount” is meant a sufficient amount of the active ingredient of the present invention to induce an immune response at a reasonable benefit/risk ratio applicable to the medical treatment.

### Antibodies:

The present invention relates to an antibody that is directed against CD40 comprising:
a heavy chain comprising the complementarity determining regions CDR1H, CDR2H and CDR3H, the CDR1H having the amino acid sequence GFTFSDYYMY (SEQ ID NO: 1), the CDR2H having the amino acid sequence YINSGGGSTYYPDTVKG (SEQ ID NO: 2), and the CDR3H having the amino acid sequence RGLPFHAMDY (SEQ ID NO: 3),
and a light chain comprising the complementarity determining regions CDR1L, CDR2L and CDR3L, the CDR1L having the amino acid sequence SASQGISNYLN (SEQ ID NO: 4) the CDR2L having the amino acid sequence YTSILHS (SEQ ID NO:5) and the CDR3L having the amino acid sequence QQFNKLPPT (SEQ ID NO:6)
   and wherein the heavy chain is fused to the polyepitope polypeptide that comprises the Ag85B epitope as set forth in SEQ ID NO:7, the ESAT-6 epitope as set forth in SEQ ID NO:8 and the Mpt64 epitope as set forth in SEQ ID NO:9.

>SEQ ID NO: 7: Ag85B epitope
SRPGLPVEYLQVPSPSMGRDIKVQFQSGGNNSPAVYLLDGLRAQDDYNGW
DINTPAFEWYYQSGLSIVMPVGGQSSFYSDWYSPACGKAGCQTYKWETFL
TSELPQWLSANRAVKPTGSAAIGLSMAGSSAMILAAYHPQQFIYAGSLSA
LLDPSQGMGPSLIGLAMGDAGGYKAADMWGPSSDPAWERNDPTQQIPKLV
ANNTRLWVYCGNGTPNELGGANIPAEFLENFVRSSNLKFQDAYNAAGGHN
AVFNFPPNGTHSWEYWGAQLNAMKGDLQSSLGAG
>SEQ ID NO: 8: ESAT-6 epitope
TSMTEQQWNFAGIEAAASAIQGNVTSIHSLLDEGKQSLTKLAAAWGGSGS
EAYQGVQQKWDATATELNNALQNLARTISEAGQAMASTEGNVTGMFA
>SEQ ID NO: 9: Mpt64 epitope
AAPKTYCEELKGTDTGQACQIQMSDPAYNINISLPSYYPDQKSLENYIAQ
TRDKFLSAATSSTPREAPYELNITSATYQSAIPPRGTQAVVLKVYQNAGG
THPTTTYKAFDWDQAYRKPITYDTLWQADTDPLPVVFPIVQGELSKQTGQ
QVSIAPNAGLDPVNYQNFAVTNDGVIFFFNPGELLPEAAGPTQVLVPRSA
IDSMLA

Anti-CD40 antibodies:

In some embodiments, the antibody is an IgG antibody, preferably of an IgG1 or IgG4 antibody, or even more preferably of an IgG4 antibody.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the heavy chain of the antibody comprises the VH domain as set forth in SEQ ID NO:10 and the light chain comprises the VL domain as set forth in SEQ ID NO:11.

>SEQ ID NO: 10: VH amino acid sequence of 12E12
humanized H3 heavy chain
EVQLVESGGGLVQPGGSLKLSCATSGFTFSDYYMYWVRQAPGKGLEWVAY
INSGGGSTYYPDTVKGRFTISRDNAKNTLYLQMNSLRAEDTAVYYCARRG
LPFHAMDYWGQGTLVTVSSAS
>SEQ ID NO: 11 VL amino acid sequence the 12E12
humanized K2 light chain
DIQMTQSPSSLSASVGDRVTITCSASQGISNYLNWYQQKPGKAVKLLIYY
TSILHSGVPSRFSGSGSGTDYTLTISSLQPEDFATYYCQQFNKLPPTFGG
GTK

In some embodiments, the heavy chain of the antibody consists of the amino acid sequence as set forth in SEQ ID NO:12 and the light chain consists of the amino acid sequence as set forth in SEQ ID NO:13.

>SEQ ID NO: 12: 12E12 humanized H3 heavy chain
EVQLVESGGGLVQPGGSLKLSCATSGFTFSDYYMYWVRQAPGKGLEWVAY
INSGGGSTYYPDTVKGRFTISRDNAKNTLYLQMNSLRAEDTAVYYCARRG
LPFHAMDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
>SEQ ID NO: 13: 12E12 humanized K2 light chain
DIQMTQSPSSLSASVGDRVTITCSASQGISNYLNWYQQKPGKAVKLLIYY
TSILHSGVPSRFSGSGSGTDYTLTISSLQPEDFATYYCQQFNKLPPTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Polyepitope polypeptides:

In some embodiments, the polyepitope polypeptide has the formula of “Ag85B-L1-ESAT-6-L2-Mpt64” wherein L1 and L2 represents a linker.

In some embodiments, the linker is selected from the group consisting of FlexV1, f1, f2, f3, or f4 as described below.

(flexV1, SEQ ID NO: 14)
QTPTNTISVTPTNNSTPTNNSNPKPNP
(f1, SEQ ID NO: 15)
SSVSPTTSVHPTPTSVPPTPTKSSP
(f2, SEQ ID NO: 16)
PTSTPADSSTITPTATPTATPTIKG
(f3, SEQ ID NO: 17)
TVTPTATATPSAIVTTITPTATTKP
(f4, SEQ ID NO: 18)
TNGSITVAATAPTVTPTVNATPSAA

In some embodiments, the polyepitope polypeptide has the formula of “Ag85B-f1-ESAT-6-f4-Mpt64” and typically consists of the amino acid sequence as set forth in SEQ ID ID: 19.

>SEQ ID NO: 19: Ag85B-f1-ESAT-6-f4-Mpt64
SRPGLPVEYLQVPSPSMGRDIKVQFQSGGNNSPAVYLLDGLRAQDDYNGW
DINTPAFEWYYQSGLSIVMPVGGQSSFYSDWYSPACGKAGCQTYKWETFL
TSELPQWLSANRAVKPTGSAAIGLSMAGSSAMILAAYHPQQFIYAGSLSA
LLDPSQGMGPSLIGLAMGDAGGYKAADMWGPSSDPAWERNDPTQQIPKLV
ANNTRLWVYCGNGTPNELGGANIPAEFLENFVRSSNLKFQDAYNAAGGHN
AVFNFPPNGTHSWEYWGAQLNAMKGDLQSSLGAGEFPTSTPADSSTITPT
ATPTATPTIKGTSMTEQQWNFAGIEAAASAIQGNVTSIHSLLDEGKQSLT
KLAAAWGGSGSEAYQGVQQKWDATATELNNALQNLARTISEAGQAMASTE
GNVTGMFAKLGGAPTNGSITVAATAPTVTPTVNATPSAASSAAPKTYCEE
LKGTDTGQACQIQMSDPAYNINISLPSYYPDQKSLENYIAQTRDKFLSAA
TSSTPREAPYELNITSATYQSAIPPRGTQAVVLKVYQNAGGTHPTTTYKA
FDWDQAYRKPITYDTLWQADTDPLPVVFPIVQGELSKQTGQQVSIAPNAG
LDPVNYQNFAVTNDGVIFFFNPGELLPEAAGPTQVLVPRSAIDSMLAEF

### Fusion:

In some embodiments, the heavy chain of the antibody is fused to the polyepitope polypeptide to form a fusion protein.

In some embodiments, the polyepitope polypeptide is fused either directly or via a linker to the heavy and/or light chain.

In some embodiments, the linker is selected from the group consisting of FlexV1, f1, f2, f3, or f4 as described above. In particular, the linker is FlexV1.

### BEST MODE

In some embodiments, the antibody of the present invention comprises the heavy chain having the amino acid sequence as set forth in SEQ ID NO:20 and the light chain having the amino acid sequence as set forth in SEQ ID NO:13 (“CD40.TB” vaccine).

>SEQ ID NO: 20: CD40.TB heavy chain
EVQLVESGGGLVQPGGSLKLSCATSGFTFSDYYMYWVRQAPGKGLEWVAY
INSGGGSTYYPDTVKGRFTISRDNAKNTLYLQMNSLRAEDTAVYYCARRG
LPFHAMDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKASQT
PTNTISVTPTNNSTPTNNSNPKPNPASSRPGLPVEYLQVPSPSMGRDIKV
QFQSGGNNSPAVYLLDGLRAQDDYNGWDINTPAFEWYYQSGLSIVMPVGG
QSSFYSDWYSPACGKAGCQTYKWETFLTSELPQWLSANRAVKPTGSAAIG
LSMAGSSAMILAAYHPQQFIYAGSLSALLDPSQGMGPSLIGLAMGDAGGY
KAADMWGPSSDPAWERNDPTQQIPKLVANNTRLWVYCGNGTPNELGGANI
PAEFLENFVRSSNLKFQDAYNAAGGHNAVFNFPPNGTHSWEYWGAQLNAM
KGDLQSSLGAGEFPTSTPADSSTITPTATPTATPTIKGTSMTEQQWNFAG
IEAAASAIQGNVTSIHSLLDEGKQSLTKLAAAWGGSGSEAYQGVQQKWDA
TATELNNALQNLARTISEAGQAMASTEGNVTGMFAKLGGAPINGSITVAA
TAPTVTPTVNATPSAASSAAPKTYCEELKGTDTGQACQIQMSDPAYNINI
SLPSYYPDQKSLENYIAQTRDKFLSAATSSTPREAPYELNITSATYQSAI
PPRGTQAVVLKVYQNAGGTHPTTTYKAFDWDQAYRKPITYDTLWQADTDP
LPVVFPIVQGELSKQTGQQVSIAPNAGLDPVNYQNFAVTNDGVIFFFNPG
ELLPEAAGPTQVLVPRSAIDSMLAEF

Methods of production:

The antibodies of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, the antibodies of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In some embodiments, the amino acid sequence herein described comprise one or more sequences originating from the restriction cloning site(s) present in the polynucleotide encoding for said amino acid sequence. Typically, said sequences may consist of 2 amino acid residues and typically include AP, AS, AR, PR, SA, TR, and TS sequences.

In some embodiments, the amino acid sequences herein described may comprise a signal peptide. As used herein, the term “signal peptide” has its general meaning in the art and refers to a pre-peptide which is present as an N-terminal peptide on a precursor form of a protein. The function of the signal peptide is to facilitate translocation of the expressed polypeptide to which it is attached into the endoplasmic reticulum. The signal peptide is normally cleaved off in the course of this process. The signal peptide may be heterologous or homologous to the organism used to produce the polypeptide.

Polynucleotides, vectors and host cells of the present invention:

A further object of the invention relates to a polynucleotide that encodes for a heavy chain and/or light chain of the antibody of the present invention.

Thus, in particular embodiment, the invention relates to a polynucleotide that encodes for a
a heavy chain fused to the polyepitope polypeptide that comprises the Ag85B epitope as set forth in SEQ ID NO:7, the ESAT-6 epitope as set forth in SEQ ID NO:8 and the Mpt64 epitope as set forth in SEQ ID NO:9, wherein the heavy chain comprises the complementarity determining regions CDR1H, CDR2H and CDR3H, the CDR1H having the amino acid sequence GFTFSDYYMY (SEQ ID NO:1), the CDR2H having the amino acid sequence YINSGGGSTYYPDTVKG (SEQ ID NO:2), and the CDR3H having the amino acid sequence RGLPFHAMDY (SEQ ID NO:3), and/or
a light chain comprising the complementarity determining regions CDR1L, CDR2L and CDR3L, the CDR1L having the amino acid sequence SASQGISNYLN (SEQ ID NO:4) the CDR2L having the amino acid sequence YTSILHS (SEQ ID NO:5) and the CDR3L having the amino acid sequence QQFNKLPPT (SEQ ID NO:6).

Typically, said polynucleotide is a DNA or RNA molecule, which may be included in any suitable vector, such as a plasmid, cosmid, episome, artificial chromosome, phage or a viral vector.

So, a further object of the invention relates to a vector comprising a polynucleotide of the present invention.

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

Such vectors may comprise regulatory elements, such as a promoter, enhancer, terminator and the like, to cause or direct expression of said antibody upon administration to a subject.

As used herein, the term “promoter/regulatory sequence” refers to a polynucleotide sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous polynucleotide sequence resulting in expression of the latter. For example, a first polynucleotide sequence is operably linked with a second polynucleotide sequence when the first polynucleotide sequence is placed in a functional relationship with the second polynucleotide sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, PAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a polynucleotide and/or a vector according to the invention. As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The polynucleotides of the invention may be used to produce an antibody of the present invention in a suitable expression system.

As used herein, the term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts. Mammalian host cells include Chinese Hamster Ovary (CHO cells) including dhfr-CHO cells (described in Urlaub and Chasin, 1980) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing the antibody according to the invention, said method comprising the steps of: (i) introducing in vitro or ex vivo a recombinant polynucleotide or a vector as described above into a competent host cell, (ii) culturing in vitro or ex vivo the recombinant host cell obtained and (iii), optionally, selecting the cells which express and/or secrete said antibody.

Such recombinant host cells can be used for the production of antibodies of the present invention.

The host cell as disclosed herein are thus particularly suitable for producing the antibody of the present invention. Indeed, when recombinant expression are introduced into mammalian host cells, the polypeptides are produced by culturing the host cells for a period of time sufficient for expression of the antibody in the host cells and, optionally, secretion of the antibody into the culture medium in which the host cells are grown. The antibodies can be recovered and purified for example from the culture medium after their secretion using standard protein purification methods.

Pharmaceutical and vaccine compositions:

The antibodies as described herein may be administered as part of one or more pharmaceutical compositions. Except insofar as any conventional carrier medium is incompatible with the antibodies of the present invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The antibodies as described herein are particularly suitable for preparing vaccine composition. Thus a further object of the present invention relates to a vaccine composition comprising an antibody of the present invention.

In some embodiments, the vaccine composition of the present invention comprises an adjuvant. In some embodiments, the adjuvant is alum. In some embodiments, the adjuvant is Incomplete Freund's adjuvant (IFA) or other oil based adjuvant that is present between 30-70%, preferably between 40-60%, more preferably between 45-55% proportion weight by weight (w/w). In some embodiments, the adjuvant is Polyinosinic-polycytidylic acid (poly (I: C)) or polyinosinic-polycytidylic acid and poly-L-lysine (poly-ICLC). In some embodiments, the vaccine composition of the present invention comprises at least one Toll-Like Receptor (TLR) agonist which is selected from the group consisting of TLR1, TLR2, TLR3, TLR4, TLR5, TLR6, TLR7, and TLR8 agonists.

Therapeutic and prophylactic methods:

The antibodies as well as the pharmaceutical or vaccine compositions as herein described are particularly suitable for inducing an immune response against Mtb and thus can be used for vaccine purposes to prevent or to treat Mtb infection

Therefore, a further object of the present invention relates to a method for vaccinating a subject in need thereof against Mtb comprising administering a therapeutically effective amount of the antibody of the present invention.

In some embodiments, the antibodies as well as the pharmaceutical or vaccine compositions as herein described are particularly suitable for the treatment of tuberculosis.

In some embodiments, the subject can be human or any other animal (e.g., birds and mammals) susceptible to Mtb infection (e.g., domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the subject can be symptomatic or asymptomatic.

In some embodiments, the vaccine of the present invention may be administered to an healthy subject or a subject at risk (immuno-suppressed through drugs, chemotherapy, immunotherapy, primary or secondary immune deficiency) to be infected by Mtb or at risk to develop TB.

Typically, the active ingredient of the present invention (i.e., the antibodies and the pharmaceutical or vaccine compositions as herein described) is administered to the subject at a therapeutically effective amount. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The antibodies and the pharmaceutical or vaccine compositions as herein described may be administered to the subject by any route of administration and in particular by oral, nasal, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active agent which is being used.

The invention will be further illustrated by the following figures and examples. However, these examples and figures should not be interpreted in any way as limiting the scope of the present invention.

### FIGURES

FIG. 1: Schematic representation of CD40.TB DC-targeting vaccine. The clone 12E12 mAb is a fully humanized anti-human CD40 IgG4 mAb. Selected Ags from Mtb, chosen from our in vitro experiments and literature, are associated, while adding flexible spacers to improve their synthesis and/or secretion.

FIG. 2: Targeting APC of active TB individuals (ATBi) via CD40 improves antigens presentation and induces specific mtb immune responses. PBMC from 6 active TB patients were stimulated with different concentrations (from 0.03 μM to 3 nM) of CD40.TB (top) or IgG4.TB control (bottom). After 8 day-culture and stimulation with 15-mer peptide pools derived from ESAT-6 or CFP-10 (not contained in the vaccine), specific CD4+ T cells producing IFNγ were analyzed (mean±SD). A. Ag, specific CD4+ T cells producing Th1, Th2 (data non shown) cytokines were analyzed (mean±SD). B. Flow cytometry dot plots are representative examples of IFNγ, TNFα, IL-2 and MIP1-81 gated on CD4+ T cells. C. Quantitative cytokines in the supernatants of PBMCs 2 days after stimulation with aCD40.TB (in blue) or IgG4.TB (in black). Results are represented as mean±SD

FIG. 3. CD40.TB vaccine elicits polyfonctional antigen-specific CD4+ T cell responses in ATBi, LTBi and in HD in vitro. Frequency of total cytokines (IFN-γ+IL-2+ TNF) produced by specific CD4+ T cells from ATBi, LTBi or HD (n=11) after in-vitro stimulation with the aCD40.TB vaccine (3 μM) on DO and re-stimulation with 15-mer peptide pool derived from ESAT-6 (1 μg/mL) and wall Ag85b and MPT64 protein (5 μg/mL) (A) or each Mtb peptide separately (C). (B) Polyfunctional composition of Mtb-specific CD4+ T-cell responses induced by the aCD40.TB vaccine. Responses are color coded according to the combination of cytokines produced. The arcs identify cytokine-producing subsets (IFN-γ, IL-2, and TNF-α) within the CD4+ T-cell populations. Median values±IQRs are shown. [Mann Whitney test] were used for comparisons (*P<0.03, *** P<0.001, **** P<0.0001).

FIG. 4. In vitro LT proliferative capacity & Cytotoxic potential. Proliferation of CD4 and CD8 T cells in PBMCs from ATBi patients (n=9) (A) or HD (n=9) (B), stimulated with aCD40.TB or IgG4.TB control. Results are represented as a proliferation index (ratio of stimulated to unstimulated cells). An index greater than 1 indicates the induction of proliferation. Results are represented as median and [Mann Whitney test] were used for comparisons (** P<0.008, **** P<0.0001). Cytotoxic potential of Mtb-specific CD4+ T-cell responses in ATBi patients (n=6). (C) Flow cytometric profiles showing CD107a expression on Mtb-specific IFN-γ-producing CD4 T cells in representative ATBI subjects (unstimulated cells (negative control) and cells stimulated with CD40.TB or IgG4.TB are shown). (D) Percentages (median with range) of IFN-Y+CD107a+ expression in total CD4 Total cells or in CD4 CFSE low T cells are shown (Mann Whitney test were performed for comparison ** P<0.02). (E) Representative flow cytometry examples and (F) cumulative analyses of the expression of Perforine, granzyme GRZB, GRZA, and CD107a on Mtb-specific IFN—Y-producing CD4 T cells after 6 days of antigen-specific in vitro T-cell expansion. (G) All the possible combinations of the different markers are shown on the pie charts, each slice corresponds to the mean proportion of Mtb-specific IFN-γ-producing CD4 T cells for a certain combination of markers identified by the respective arcs.

FIG. 5. In vivo immunogenicity of CD40.TB. A. hCD40Tg mice (n=3-5/group) were immunized twice with 1 μg of CD40.TB±poly-ICLC or PBS (Mock). B. The immunogenicity of CD40.TB vaccine in vivo was tested 1 week after the 2nd immunization for its induction of both humoral (upper panels) and T cell response (lower panels) in sera and spleen, respectively. Specific Mtb Ags antibodies secreted in serum were measured by Luminex (upper panelq). Results are represented as median+IR.

### EXAMPLE

After decades of research, there is still an urgent need to accelerate the development of a potent TB vaccine according to recent WHO recommendation. Only few protein/adjuvant vaccine candidates are currently under clinical development. To date “classic” strategies failed or are not optimal and one of the top priorities of the WHO includes innovative approaches to develop new TB vaccine strategies, drugs and other health technologies to cut the risk of TB disease in the approximately 2 billion people already infected. We strongly believe that a guarantee of success is to diversify the tools and technologies and that is why we would like to exploit the DC targeting strategy by using a humanized anti-human CD40 mAb fused to relevant Mtb Ags.

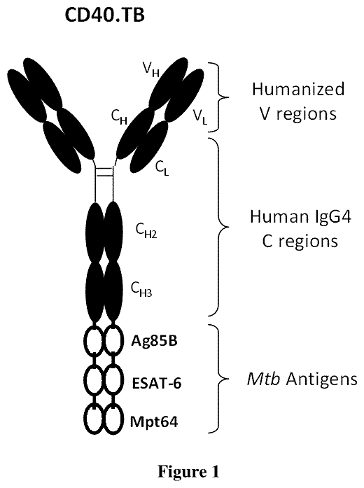

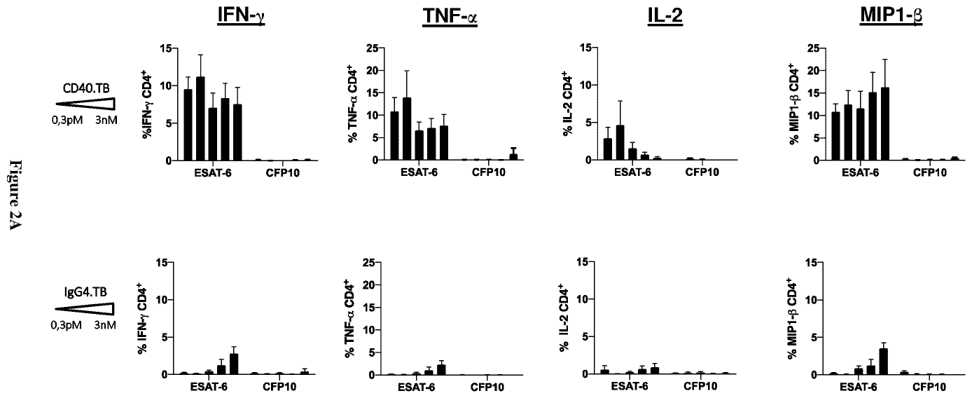

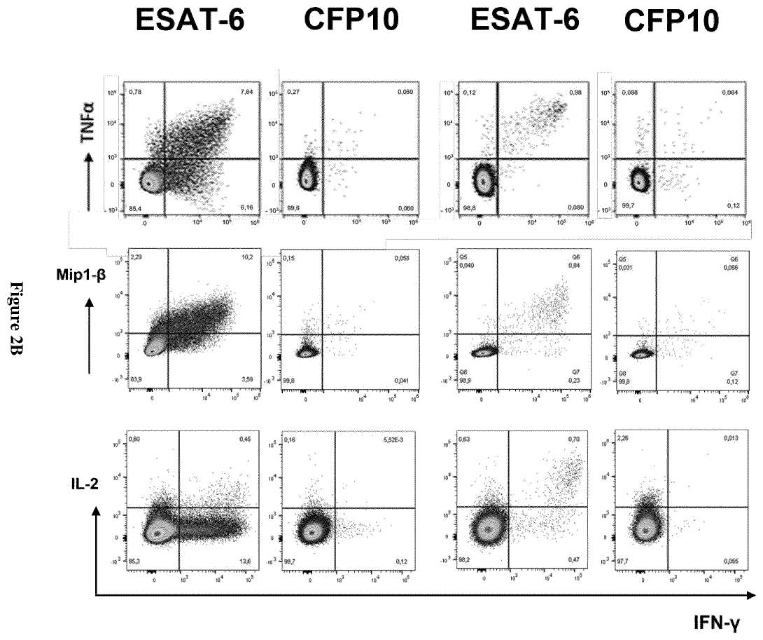

We have produced a new TB vaccine candidate by directly fusing three Mtb Ags, namely ESAT-6, Ag85B and Mpt64, to the C-terminus end of the heavy chains of the 12E12 mAb specific to human CD40 (“CD40.TB construct”) (FIG. 1).

ESAT-6 is one of the most immunogenic Ags of Mtb. This Ag is part of a number of live attenuated and subunit vaccines currently being tested for TB, including MTBVAC [48], H1 and GamTBvac but is absent from the BCG.

Nearly ten current TB vaccine candidates contain Ags of the Ag85 complex, namely Ag85A or Ag85B. In particular, Ag85B contains several immunodominant T cell epitopes and Ags of the Ag85 complex are part of several vaccine candidates currently being tested for TB. In particular, BCG over-expressing Ag85B confers greater protection against Mtb than BCG in guinea pigs [51], and DNA vaccination with plasmids encoding Ag85B conferred robust Th1 immunity and protection in mice [52]. Moreover, several Ag85B epitopes are recognized by human CD4+ and CD8+ T cells from PPD+ individuals [53-57].

Mpt64 is a secreted protein encoded in the region of difference RD2, which is absent from “late” BCG strains, such as the most widely used Pasteur and Danish BCG subtypes [59]. It has been shown that DNA vaccine including Ag85B and Mpt64 Ags, along with conventional TB chemotherapy, could be effective in the prevention of TB reactivation and a promising strategy for controlling Mtb infection in mice [60]. Mpt64 and ESAT-6 are also contained in the H107 vaccine associated with a substantial increase in long-term protection [27].

CD40.TB constructs have been tested in transiently transfected 293F cells and selected for stable transfection of a CHO—S cell line. Antibodies produced in supernatants have been purified, quality-controlled, and tested for binding to mononuclear primary cells from healthy donors by flow cytometry. We also generated a negative control of targeting, an IgG4 Ab fused to the same Mtb Ags, referred to IgG4.TB.

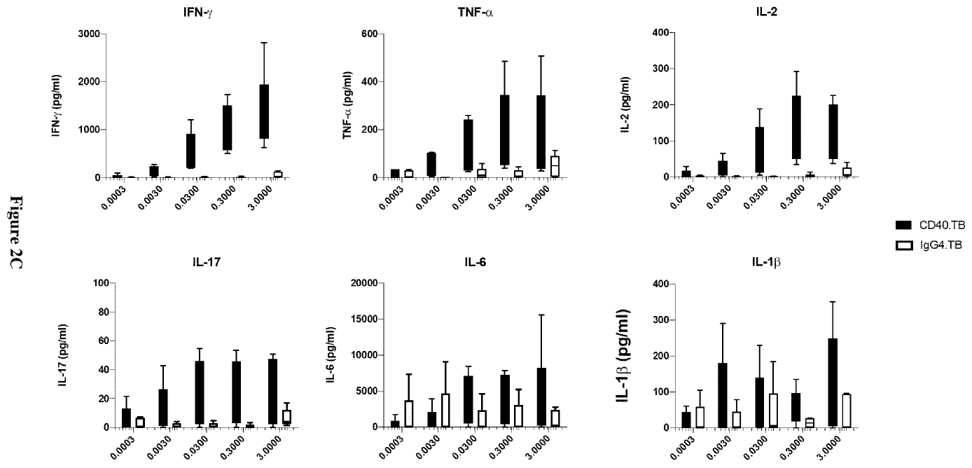

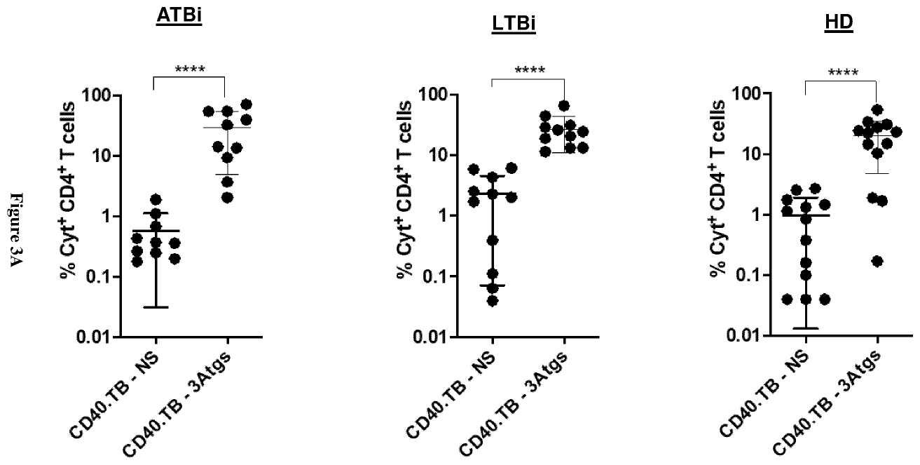

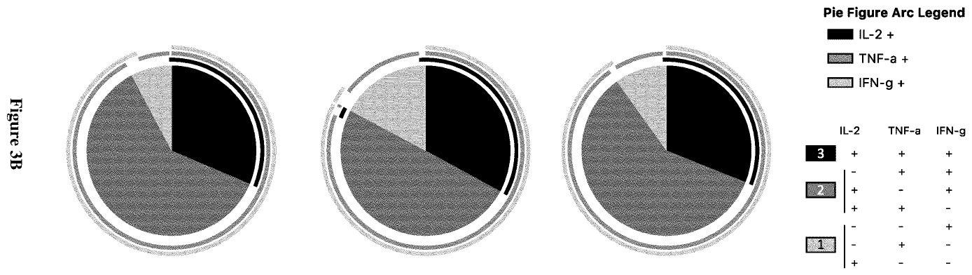

We investigated the capacity of CD40.TB to elicit Mtb-specific T cell responses by assessing the characteristics of recall and de novo CD4+ and CD8+ T cell responses in vitro in PBMC from 6 active TB patients (ATBi), as previously done with a CD40.SARS-COV-2 vaccine with PBMC from convalescent COVID-19 individuals (FIG. 2). Human PBMC were cultured with various concentrations of CD40.TB or IgG4.TB and restimulated with recombinant proteins or pools of 15-mer peptides covering the entire sequences of ESAT-6 or CFP-10 Ags (BEI Resources, NIH). As shown in FIG. 2A-2B, Mtb-specific CD4+ T cell responses are drastically improved upon cell stimulation with CD40.TB vaccine compared to IgG4.TB control. The difference is even more striking using very low concentration of CD40.TB construct. As expected, only very low responses are observed when cells were restimulated with CFP-10, an Ag not included in the CD40.TB vaccine. CD40.TB vaccine induced Th1 and pro-inflammatory cytokines such as MIP1-B, IL-1ß and IL-6, as well as IL-17 cytokine secretion (FIG. 2C).

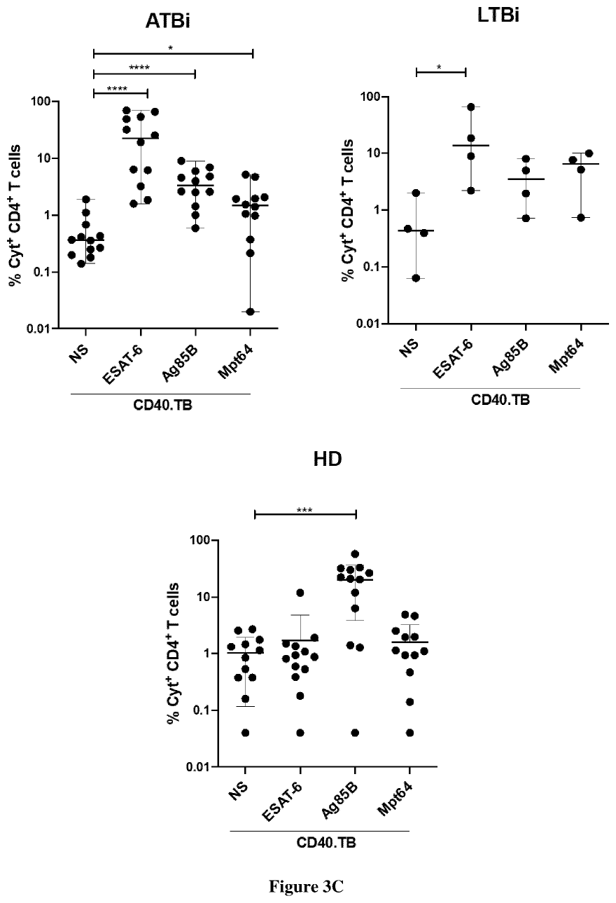

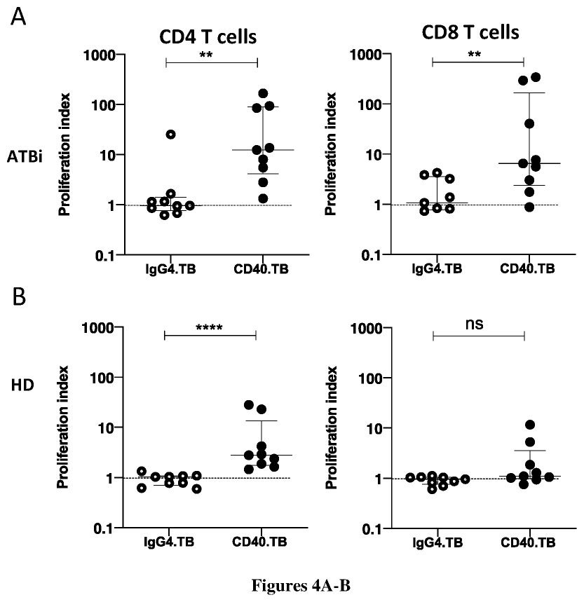

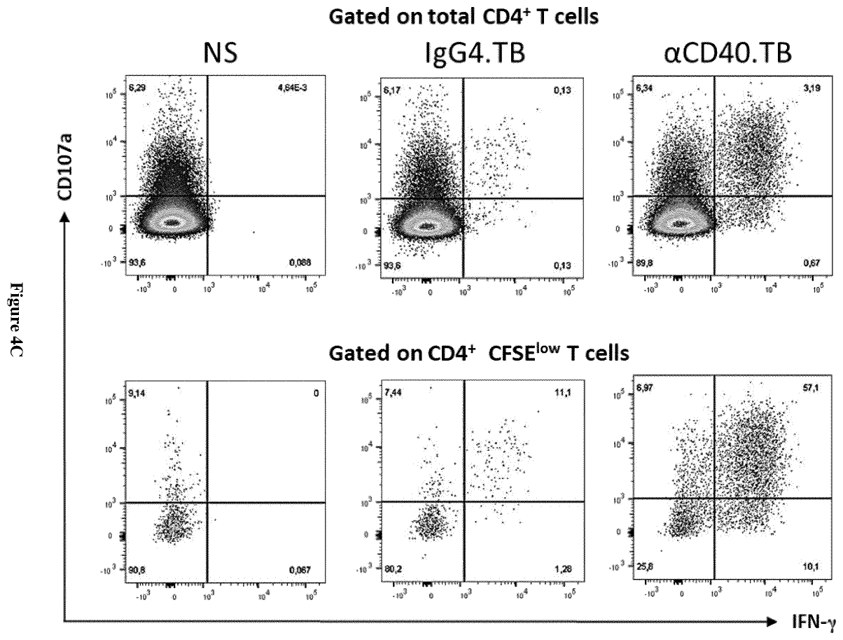

We investigated the immunogenicity of the 3 Mtb antigens contained in the vaccine (FIG. 3). After 8 days of culture with 3 μmol/l the vaccine, ATBi, LTBi (latent TB individuals) and HD (healthy BCG vaccinated donors from Etablissement Français du Sang) PBMCs were restimulated with the pool of Mtb 15-mer peptide ESAT6, wall Ag85b and MPT64 protein or each peptide separately. On day 9, CD4+ producing Th1 (IFN-γ, TNF-α, IL-2), Th2 (IL-10, IL-4, IL-13) and MIP1-β cytokines were analyzed by flow cytometer. In ATBi, LTBi and HD, there was an increase in the frequency of Th1 cytokine (IFN-γ, TNF-α and IL-2) producing CD4+ T cells after restimulation with the 3 antigens compared to non-stimulated cells (FIG. 3A). The vaccine is therefore able to induce recall responses by Mtb-specific CD4+ T cells in both groups of individuals. Furthermore, using Boolean gating to examine multiple cytokine combinations at the single cell level (i.e., polyfunctional responses), CD4+T polyfunctionality analyses show that the predominant cytokine combinations in responses to the 3 Mtb antigens stimulation were bi-functional, producing simultaneously 2 cytokines followed by tri-functional Mtb-specific CD4+ T cells simultaneously producing up to three cytokines (IFN-γ+IL-2±TNF) (FIG. 3 B). Cells producing only one cytokine were less prevalent in both 3 groups. Regarding the capacity of each Mtb antigens to expand memory cells individually, we observed that, ESAT-6 responses were predominantly induced in both ATBi (22.44%) and LTBi (13.74%), followed by Ag85b and MPT64 (3.31% and 1,4%) respectively in ATBi (3.489% and 6,451%) respectively in LTBi. Whereas the response to Ag85b was the most and the only prevalent with 20.93% of total CD4+ T cells producing cytokines in HD (FIG. 3C). Taken together, these results demonstrate the ability of the CD40.TB vaccine to stimulate specific memory cells for the three Mtb antigens in both ATBi and LTBi, while in HD only Ag85b specific memory cells were expanded.

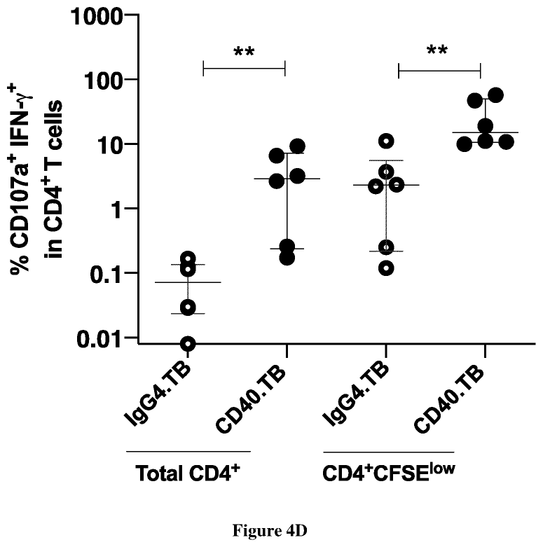

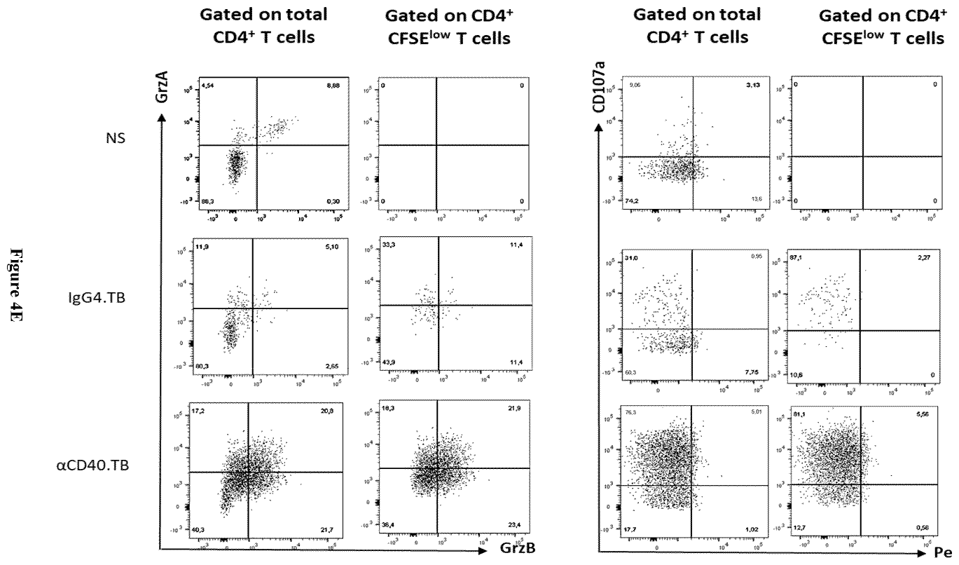

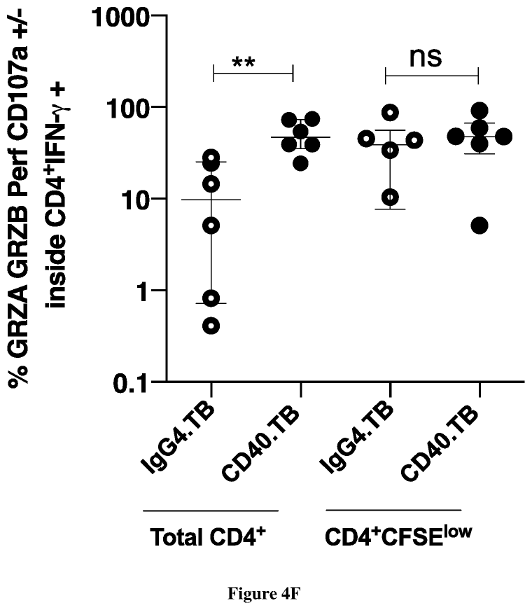

Another important feature is that CD8+ T cells have a proliferative capacity induced by CD40.TB vaccine as shown in FIG. 4, although at a lesser extent than CD4+ T cells. The frequency of proliferative T cells is higher upon cell stimulation with CD40.TB vaccine compared to IgG4.TB (FIG. 4A). Stimulated HD PBMCs with CD40.TB vaccine also induces CD4+ and CD8+ T cell proliferation (FIG. 4B). We also evaluated whether Mtb Ag-specific T cells may display cytotoxic properties, degranulation after CD40.TB vaccine stimulation for 6 days and overnight restimulation with 3 Mtb antigens present in CD40.TB vaccine. We evaluate first of all the lysosomal-associated membrane glycoprotein CD107a and IFN-γ co-expression at the surface of Mtb antigens induced CD4+ Total cells or in CD4+ CFSE low T cells (FIG. 4C). Mtb-specific CD4+ T cells expressing both the cytotoxicity markers CD107a and IFN-γ after peptide stimulation were also detected at high frequency in both CD4 Total cells and CD4 CFSE low cells indicating that these cell subsets had indeed both Th1 and cytotoxic characteristics (FIG. 4D). We did not detect significant specific responses against non targeting control (IgG4.TB) compared to non-stimulated cells. We characterized among specific IFN-γ CD4+ T cells, degranulation markers CD107a and cytotoxic markers such as granzyme A (GRZA), granzyme B (GRZB) and perforin (Perf). Cytometry dot plots from one representative individual showing cytotoxic profile of Mtb-specific IFN-γ+CD4+ T cells stimulated with Mtb peptides are represented in FIG. 4E. We observed that IFN-γ+CD4+ T-cells induced by CD40.TB vaccine were mainly polycytotoxic expressing CD107a, GRZA, GRZB or Perf importantly compared to unstimulated cells or to IgG4.TB control (FIGS. 4F and 4G)

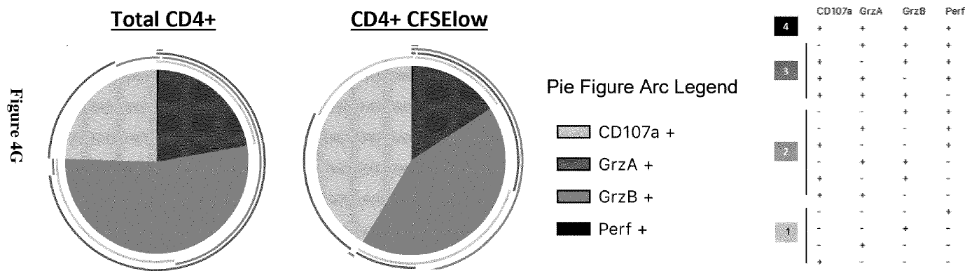

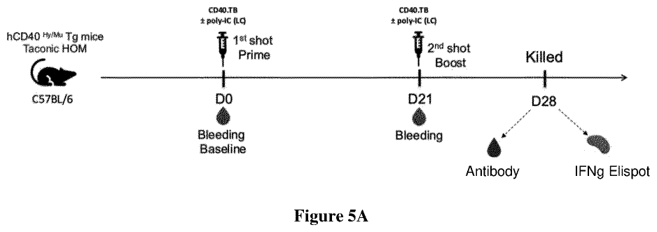

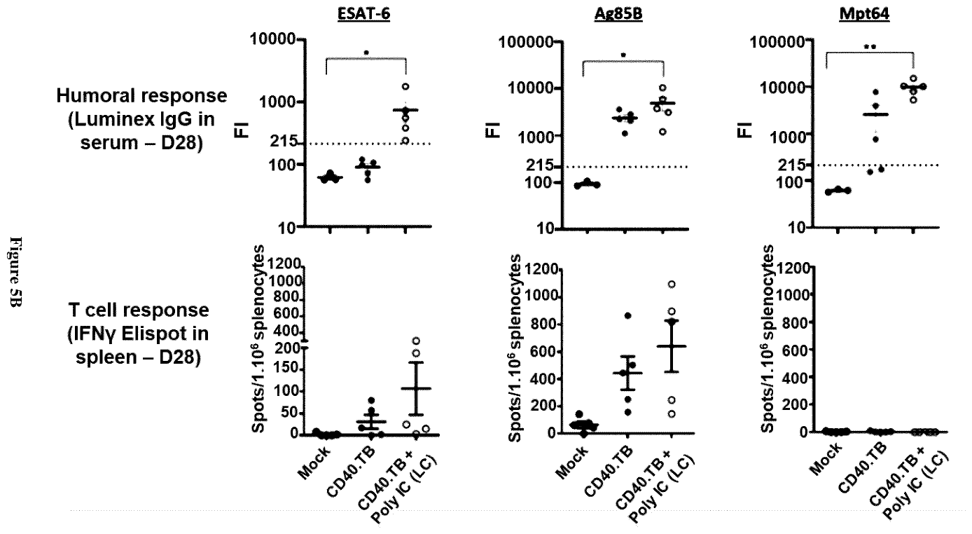

Finally two immunizations of hCD40Tg mice at day 0 and day 21 with lug/mouse of CD40.TB vaccine in the presence or absence of poly-ICLC induce Mtb-specific humoral and T cell responses against the 3 Ags contained in the CD40.TB vaccine compared to non-immunized mice (FIG. 5).

### REFERENCES

Throughout this application, various references describe the state of the art to which this invention pertains. The disclosures of these references are hereby incorporated by reference into the present disclosure.
1. Dheda K, Lancet. 2016; PMID: 26377143.
2. Zumla A, Lancet Infect Dis. 2013; PMID: 23531385.
3. Conradie F, N Engl J Med. 2020; PMID: 32130813.
4. Morrison J, Lancet Infect Dis. 2008; PMID: 18450516.
5. da Costa C, Int J Infect Dis. 2015; PMID: 25809749.
6. Jeyanathan M, Trends Immunol. 2018; PMID: 29429859.
7. Shaler C R, Front Immunol. 2013; PMID: 23420646.
8. Jasenosky L D, Immunol Rev. 2015; PMID: 25703553.
9. Rozot V, Clin Infect Dis. 2015; PMID: 25362202.
10. Marin N D, Tuberculosis. 2013; PMID: 23332142.
11. Berry M P, Nature. 2010; PMID: 20725040.
12. Cliff J M, Immunol Rev. 2015; PMID: 25703554.
13. Gupta R K, Lancet Respir Med. 2020; PMID: 31958400.
14. Schrager L K, Lancet Infect Dis. 2020; PMID: 32014117.
15. Kaufmann S H E, Front Immunol. 2017; PMID: 29046674.
16. Andersen P and Scriba T J, Nat Rev Immunol. 2019; PMID: 31114037.
17. Cardona P J, Front Microbiol. 2016; PMID: 27733848.
18. Cardona P J, Tuberculosis. 2006; PMID: 16545981.
19. Majlessi L, Immunol Rev. 2015; PMID: 25703550.
20. Nayak K, Tuberculosis. 2015; PMID: 25857935.
21. Bettencourt P, Vaccines. 2020; PMID: 31908851.
22. Groschel M I, Nat Rev Microbiol. 2016; PMID: 27665717.
23. Sayes F, Cell Rep. 2018; PMID: 29694886.
24. Sayes F, Cell Host Microbe. 2012; PMID: 22520463.
25. Shanley C A, Tuberculosis. 2014; PMID: 24295653.
26. Coler R N, J Infect Dis. 2013; PMID: 22891286.
27. Woodworth J S, Nat Commun. 2021; PMID: 34795205.
28. Lowrie D B, Vaccine. 2006; PMID: 16316711.
29. Tameris M D, Lancet. 2013; PMID: 23391465.
30. Hansen S G, Nat Med. 2018; PMID: 29334373.
31. Tkachuk A P, Vaccines 2020; PMID: 33153191.
32. Tait D R, N Engl J Med. 2019; PMID: 31661198.
33. Mellman I and Steinman R M, Cell. 2001; PMID: 11509172.
34. Flynn B J, Proc Natl Acad Sci USA. 2011; PMID: 21467219.
35. Trumpfheller C, J Exp Med. 2006; PMID: 16505141.
36. Cheong C, Blood. 2010; PMID: 20668230.
37. Epaulard O, J Immunol. 2014; PMID: 25057007.
38. Cheng L, J Clin Invest. 2018; PMID: 30148455.
39. Flamar A L, PLOS One. 2018; PMID: 30500852.
40. Flamar A L, AIDS. 2013; PMID: 23615121.
41. Flamar A L, J Immunol. 2012; PMID: 22865916.
42. Li D, J Exp Med. 2012; PMID: 22213806.
43. Zurawski G, J Virol. 2017; PMID: 28202751.
44. Zurawski G, PLOS One. 2016; PMID: 27077384.
45. Marlin R, Nat Commun. 2021; PMID: 34471122.
46. Coléon S, ebiomed 2022; in revision.
47. Yin W, Cancer Immunol Res. 2016; PMID: 27485136.
48. Marinova D, Exp Rev Vaccines. 2017; PMID: 28447476.
49. Mearns H, Vaccine. 2017; PMID: 27866772.
50. Huygen K, Front Immunol. 2014; PMID: 25071781.
51. Horwitz M A, Proc Natl Acad Sci USA. 2000; PMID: 11095745.
52. Lozes E, Vaccine. 1997; PMID: 9234526.
53. Silver R F, J Immunol. 1995; PMID: 7722319.
54. Valle M T, Clin Exp Immunol. 2001; PMID: 11207652.
55. Mustafa A S, Infect Immun. 2000; PMID: 10858206.
56. Lindestam Arlehamn C S, PLOS Pathog. 2013; PMID: 23358848.
57. Weichold F F, Genes Immun. 2007; PMID: 17429413.
58. Stamm C E, mSphere. 2019; PMID: 31167949.
59. Brosch R, Proc Natl Acad Sci USA.2007; PMID: 17372194.
60. Yu D H, Gene Ther. 2008; PMID: 18288210.
61. Flynn J L, Immunol Rev. 2015; PMID: 25703552.
62. Singh A K and Gupta D U, Indian J Med Res. 2018; PMID: 30082569.
63. Pym A S, Nat Med. 2003; PMID: 12692540.
64. Groschel M I, Cell Rep. 2017; PMID: 28297677.
65. Leung-Theung-Long S, PLOS One 2018; PMID: 29718990.
66. Scanga C A, Cold Spring Harb Perspect Med. 2014; PMID: 25213189.
67. Capuano S V, Infect Immun. 2003; PMID: 14500505.
68. Lin P L, Nat Med. 2014; PMID: 24336248.
69. Sharpe S A, PLOS One. 2017; PMID: 28273087.
70. Sharpe S, Tuberculosis. 2016; PMID: 27865390.
71. Sharpe S A, Clin Vaccine Immunol. 2010; PMID: 20534795.
72. Druett H A, J Hyg (Lond). 1969; PMID: 5258223.
73. Mattila J T, J Immunol. 2017; PMID: 28592427.
74. Roberts L, Nature 2022; PMID: 35292767.

1. An antibody that is directed against CD40 comprising:
a heavy chain comprising the complementarity determining regions CDR1H, CDR2H and CDR3H, the CDR1H having the amino acid sequence GFTFSDYYMY (SEQ ID 5 NO: 1), the CDR2H having the amino acid sequence YINSGGGSTYYPDTVKG (SEQ ID NO:2), and the CDR3H having the amino acid sequence RGLPFHAMDY (SEQ ID NO:3),
and a light chain comprising the complementarity determining regions CDR1L, CDR2L and CDR3L, the CDR1L having the amino acid sequence SASQGISNYLN (SEQ ID 10 NO: 4) the CDR2L having the amino acid sequence YTSILHS (SEQ ID NO: 5) and the CDR3L having the amino acid sequence QQFNKLPPT (SEQ ID NO: 6)
and wherein the heavy chain is fused to the polyepitope polypeptide that comprises the Ag85B epitope as set forth in SEQ ID NO:7, the ESAT-6 epitope as set forth in SEQ ID NO:8 and the Mpt64 epitope as set forth in SEQ ID NO:9.

2. The antibody of the claim 1 wherein the heavy chain of the antibody comprises the VH domain as set forth in SEQ ID NO:10 and/or the light chain comprises the VL domain as set forth in SEQ ID NO:11.

3. The antibody of claim 2 wherein the heavy chain of the antibody consists of the amino acid sequence as set forth in SEQ ID NO:12 and/or the light chain consists of the amino acid sequence as set forth in SEQ ID NO:13.

4. The antibody according to claim 1, wherein the polyepitope polypeptide has a formula: Ag85B-L1-ESAT-6-L2-Mpt64, wherein L1 and L2 each represents a linker.

5. The antibody of claim 4 wherein the L1 and/or L2 is selected from the group consisting of SEQ ID NO:14 (Flex V1), SEQ ID NO:15 (f1), SEQ ID NO:16 (f2), SEQ ID NO:17 (f3), and SEQ ID NO: 18 (f4).

6. The antibody of claim 5 wherein the polyepitope polypeptide has a formula: “Ag85B-f1-ESAT-6-f4-Mpt64”.

7. The antibody of claim 6 wherein the polyepitope polypeptide consists of the amino acid sequence as set forth in SEQ ID ID: 19.

8. The antibody according to claim 1, wherein the heavy chain of the antibody is fused to the polyepitope polypeptide to form a fusion protein.

9. The antibody of claim 8 wherein the polyepitope polypeptide is fused either directly or via a linker to the heavy chain.

10. The antibody of claim 9 wherein the linker is FlexV1 (SEQ ID NO: 14).

11. The antibody according to claim 1, wherein the heavy chain has the amino acid sequence as set forth in SEQ ID NO:20 and the light chain has the amino acid sequence as set forth in SEQ ID NO:13.

12. A polynucleotide that encodes for the heavy chain and/or the light chain of the antibody according to claim 1.

13. A vector comprising the polynucleotide of claim 12.

14. A host cell which has been transfected, infected or transformed by the polynucleotide of claim 12 and/or by a vector comprising the polynucleotide.

15. A vaccine composition comprising the antibody according to claim 1.

16. A method for vaccinating a subject in need thereof against Mycobacterium tuberculosis comprising administering to the subject a therapeutically effective amount of the antibody according to claim 1.
